(19) United States
(12) Patent Application Publication
(10) Pub. No.: US 2005/0026940 A1
(43) Pub. Date: Feb. 3, 2005

(54) Processes for the preparation of substituted bicyclic derivatives

(75) Inventors: David H. B. Ripin, Old Saybrook, CT (US); Michael G. Vetelino, North Stonington, CT (US); Lulin Wei, Salem, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/821,906

(22) Filed: Apr. 9, 2004

Related U.S. Application Data
(60) Provisional application No. 60/516,860, filed on Nov. 3, 2003.
(60) Provisional application No. 60/461,632, filed on Apr. 9, 2003.

(51) Int. Cl. A61K 31/517; A61K 31/55; C 07D 4 3/02

(52) U.S. Cl. 514/266.2; 514/266.4; 544/284; 544/293

(57) ABSTRACT

The invention relates to processes for preparing compounds of the formula 1 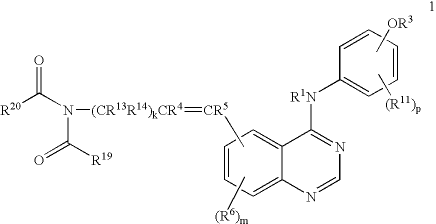
and to pharmaceutically acceptable salts, prodrugs and solvates thereof, wherein R¹, R³, R⁴, R⁶, R¹¹, R¹³, R¹⁴, R¹⁵, R¹⁶, R¹⁷, k, l, and m are as defined herein. The compounds of formula 1 are useful intermediates toward preparing compounds that may be used in treating abnormal cell growth in mammals by administering pharmaceutical compositions.

This application claims priority from U.S. Provisional Application No. 60/461,632 filed Apr. 9, 2003 and from U.S. Provisional Application No. 60/516,860 filed Nov. 3, 2003.

### BACKGROUND OF THE INVENTION

This invention relates to novel processes and intermediates useful for the preparation of substituted bicyclic derivatives. The substituted bicyclic derivatives of the present invention may be converted into compounds that are useful in the treatment of abnormal cell growth, such as cancer, in mammals and are described in International Patent Publication WO 01/98277, published Dec. 27, 2001, the contents of which are hereby incorporated by reference in its entirety.

A process for the preparation of substituted bicyclic derivatives has also been disclosed in U.S. Provisional Application Ser. No. 60/334,647 (filed Nov. 30, 2001), and in U.S. application Ser. No. 10/307,603 (filed Dec. 2, 2002), both of which are incorporated herein by reference in their entirety.

### SUMMARY OF THE INVENTION

The present invention relates to a method for preparing a compound of formula 1 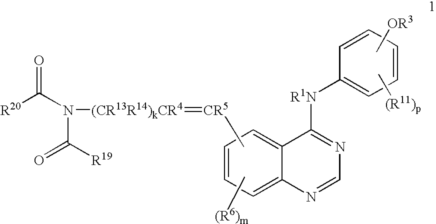
acceptable salts, and solvates thereof, wherein:
k is an integer from 1 to 3;
m is an integer from 0 to 3;
p is an integer from 0 to 4;
R¹, R², R⁴, and R⁵ are each independently selected from H and C₁-C₆ alkyl;

R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond when t is an integer between 2 and 5, and the foregoing R³ group, including any optional fused ring referred to above, is optionally substituted by 1 to 5 R¹⁰ groups;
each R⁶ is independently selected from halo, hydroxy, —NR¹R², C₁-C₆ alkyl, trifluoromethyl, C₁-C₆ alkoxy, trifluoromethoxy, —NR⁷C(O)R¹, —C(O)NR⁷R⁹, —SO₂NR⁷R⁹, —NR⁷C(O)NR⁹R¹, and —NR⁷C(O)OR⁹;
each R⁷, R⁸ and R⁹ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁷, R⁸ and R⁹ groups are optionally substituted with 1 to 3 substituents independently selected from halo, cyano, nitro, —NR¹R², trifluoromethyl, trifluoromethoxy, C₁-C₆ alkyl, C₂-C⁶ alkenyl, C₂-C₆ alkynyl, hydroxy, and C₁-C₆ alkoxy;
or each R⁷ and R⁹, or R⁸ and R⁹, when attached to a nitrogen atom, can be taken together to form a 4 to 10 membered heterocyclic ring which may include 1 to 3 additional hetero moieties, in addition to the nitrogen to which said R⁷, R⁸, and R⁹ are attached, selected from N, N(R¹), O, and S, provided two O atoms, two S atoms or an O and S atom are not attached directly to each other;
each R¹⁰ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁷, —C(O)OR⁷, —OC(O)R⁷, —NR⁷C(O)R⁹, —NR⁷SO₂NR⁹R¹, —NR⁷C(O)NR¹R⁹, —NR⁷C(O)OR⁹, —C(O)NR⁷R⁹, —NR⁷R⁹, —NR⁷OR⁹, —SO₂NR⁷R⁹, —S(O)ⱼ(C₁-C₆alkyl) wherein j is an integer from 0 to 2, 13 (CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is and interger from 0 to 2, q and t are each independently an integer from 0 to 5, 1 or 2 ring carbon atoms of the heterocyclic moieties of the foregoing R¹⁰ groups are optionally substituted with an oxo (═O) moiety, and the alkyl, alkenyl, alkynyl, aryl and heterocyclic moieties of the foregoing R¹⁰ groups are optionally substituted with 1 to 3 substituents independently selected from halo, cyano, nitro, trifluoromethyl, trifluoromethoxy, azido, —OR⁷, —C(O)R⁷, —C(O)OR⁷, —OC(O)R⁷, —NR⁷C(O)R⁹, —C(O)NR⁷R⁹, —NR⁷R⁹, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
each R¹¹ is independently selected from halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁷, —C(O)OR⁷, —OC(O)R⁷, —NR⁷C(O)R⁹, —NR⁷SO₂NR⁹R¹, —NR⁷C(O)NR¹R⁹, —NR⁷C(O)OR⁹, —C(O)NR⁷R⁹, —NR⁷R⁹, —NR⁷OR⁹, —SO₂NR⁷R⁹, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is an integer from 0 to 2, q and t are each independently an integer from 0 to 5, 1 or 2 ring carbon atoms of the heterocyclic moieties of the foregoing R¹⁰ groups are optionally substituted with an oxo (═O) moiety, and the alkyl, alkenyl, alkynyl, aryl and heterocyclic moieties of the foregoing R¹⁰ groups are optionally substituted with 1 to 3 substituents independently selected from halo, cyano, nitro, trifluoromethyl, trifluoromethoxy, azido, —OR⁷, —C(O)R⁷, —C(O)OR⁷, —OC(O)R⁷, —NR⁷C(O)R⁹, —C(O)NR⁷R⁹, —NR⁷OR⁹, C₁-C₆ alkyl, C₂-C₆ alkenyl, C₂-C₆ alkynyl, —(CR¹R²)ₜ(C₆-C₁₀ aryl), and —(CRR²)ₜ(4 to 10 membered heterocyclic), wherein t is an integer from 0 to 5;
each R¹³ and R¹⁴ are independently selected from H, C₁-C₆ alkyl, and —CH₂OH;
R¹⁹ and R²⁰ are independently selected from the group consisting of —(CR¹⁵R¹⁶)ₗOR¹⁷ and OR¹⁸ wherein each R¹⁵ and R¹⁶ is independently selected from H, C₁-C₆ alkyl, and —CH₂OH, l is an integer from 1 to 3, R¹⁷ is C₁-C₆ alkyl, R¹⁸ independently is C₁-C₆ alkyl, provided both R¹⁹ and R²⁰ are not simultaneously —(CR¹⁵R¹⁶)ₗOR¹⁷;
wherein each carbon not bound to a N or O atom, or to S(O)ⱼ, wherein j is an integer from 0 to 2, is optionally substituted with R¹², wherein R¹² is R⁷, —OR⁷, —OC(O)R⁷, —OC(O)NR⁷R⁹, —OCO₂R⁷, —S(O)ⱼR⁷, —S(O)ⱼNR⁷R⁹, —NR⁷R⁹, —NR⁷C(O)R⁹, —NR⁷SO₂R⁹, —NR⁷C(O)NR⁸R⁹, —NR⁷SO₂NR⁸R⁹, —NR⁷CO₂R⁹, CN, —C(O)R⁷, or halo, wherein j is an integer from 0 to 2; and wherein any of the above-mentioned substituents comprising a CH₃ (methyl), CH₂ (methylene), or CH (methine) group, which is not attached to a halogen, SO or SO₂ group or to a N, O or S atom, is optionally substituted with a group selected from hydroxy, halo, C₁-C₄ alkyl, C₁-C₄ alkoxy and —NR¹R²; which comprises reacting a compound of formula 2 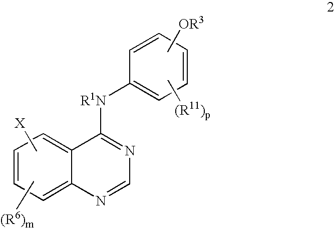
   wherein X is a halide and R¹, R³, R⁶, R¹¹, m and p are as defined for formula 1 above, with a compound of formula 3 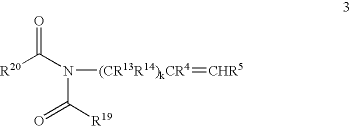
   wherein R⁴, R⁵, R¹³, R¹⁴, R¹⁹, R²⁰, and k are as defined for formula 1 above, in the presence of a catalyst, a base, and an optional ligand.

The present invention also relates to a method for preparing the aforementioned compound of formula 1, pharmaceutically acceptable salts, solvates and prodrugs thereof, which comprises reacting a compound of formula 7 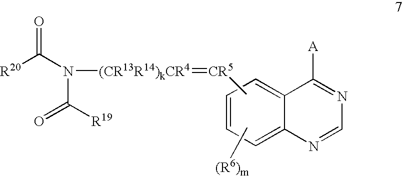
wherein A is Cl or F and R⁴, R⁵, R⁶, R¹³, R¹⁴, R¹⁹, R²⁰, k and m are as defined for formula 1 with a compound of formula 8 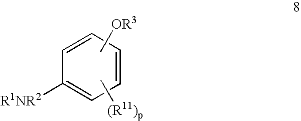
wherein R¹, R², R³, R¹¹ and p are as defined for formula 1.

In one preferred embodiment of the present invention, X in formula 2 above is a halide selected from the group consisting of chloride, bromide and iodide.

In a preferred embodiment of the invention, the catalyst is a palladium or nickel catalyst selected from the group consisting of Palladium on carbon (Pd/C), Pd(OAc)₂, Pd₂(dba)₃, PdCl₂, Pd(MeCN)₂Cl₂, Pd(PhCN)₂Cl₂, PdCl₂(PPh₃)₂, Pd(PPh₃)₄, BnPdCl(PPh₃)₂, Pd(Otfa)₂, Pd(PPh₃)₂(Otfa)₂, PdCl₂(dppf), Pd(acac)₂, Pd₂(dba)₃-CHCl₃, Ni(PPh₃)₄, Pd(dppb), trans-di(μ-acetato)-bis[o-(di-o-tolylphosphino)benzyl]dipalladium(II), bis(1,3-dihydro-1,3-dimethyl-2H-imidazol-2-ylidene)diiodo-palladium, and diiodo[methylenebis[3-(2-methyl)-1H-imidazol-1-yl-2(3H)-ylidene]]-palladium.

In a more preferred embodiment of the process for preparing the compounds of formula 1, the palladium catalyst is selected from the group consisting of Palladium on carbon (Pd/C), Pd(OAc)₂, Pd₂(dba)₃, and Pd(PPh₃)₄.

In another more preferred embodiment of the process for preparing the compounds of formula 1, the palladium catalyst is selected from the group consisting of Palladium on carbon (Pd/C), Pd(OAc)₂ and Pd(PPh₃)₄.

In a most preferred embodiment of the present invention, the catalyst is a palladium on carbon (Pd/C) catalyst. Several types of Pd/C have been found to be useful for the present invention. A variety of Pd/C loadings (such as 5% Pd/C-10% Pd/C) can be used; dry or wet catalyst can be used. Furthermore, catalyst levels of 0.25% Pd or even lower can be used in the present invention. Furthermore, these (Pd/C) catalysts are cheaper, more readily available, and easier to purge following the reaction than the other catalysts mentioned herein.

In a preferred embodiment of this process, the optional ligand is selected from the group consisting a polymer bound phosphine, BINAP, dppf, 2-methyl-2′-(dicyclohexylphosphino)biphenyl, 2-dimethylamino-2′-(dicyclohexylphosphino)biphenyl, and P(R²²)₃, wherein each R²² is independently selected from the group consisting of 2-methyl-2′-(dicyclohexylphosphino)biphenyl, 2-dimethylamino-2′-(dicyclohexylphosphino)biphenyl, phenyl, o-toluyl, OMe, and furyl.

In a more preferred embodiment of this processes of the present invention the ligand is selected from the group consisting of PPh₃, P(o-tol)₃, P(o-OMePh)₃, P(2-furyl)₃, BINAP, and dppf.

In a most preferred embodiment of the processes of the present invention the ligand is selected from the group consisting of PPh₃, P(o-tol)₃, and P(2-furyl)₃.

In a preferred embodiment of the process for preparing the compounds of formula 1, the base is selected from the group consisting of (R)₃N, (R)₂NH, RNH₂, QX, Q₂CO₃, Q₃PO₄, QO₂CR, wherein Q is selected from the group consisting of (R)₄N, Na, K, Cs, Cu, Cd, and Ca, and wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

In another preferred embodiment of the process for preparing the compounds of formula 1, the base is selected from the group consisting of R₄NF, R₄NCl, R₄NBr, Et₃N, Me₂NEt, iPr₂NEt, CuBr, Cul, CdCl, CsF, K₂CO₃, Na₃PO₄, Na₂HPO₄, NaOAc, DABCO, and 1,8-(dimethylamino)naphthalene, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

In a more preferred embodiment of the process for preparing the compounds of formula 1, the base is selected from the group consisting of Et₃N, Me₂NEt, iPR₂NEt, CuBr, Cul, CdCl, CsF, R₄NF, R₄NCl, R₄NBr, K₂CO₃, Na₃PO₄, Na₂HPO₄, NaOAc, DABCO, and 1,8-(dimethylamino)napthalene, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

In an even more preferred embodiment of the process for preparing the compounds of formula 1, the base is selected from the group consisting of Et₃N, Me₂NEt, K₂CO₃, Na₃PO₄ and NaOAc.

In a preferred embodiment of the process for preparing the compounds of formula 1, the reaction of compounds 2 and 3 is carried out in a solvent selected from the group consisting of toluene, benzene, xylene, dimethylformamide, dimethylacetamide, dioxane, tetrahydrofuran, acetonitrile, N-methylpyrrolidinone, dimethylsulfoxide, dimethoxyethane, CH₂Cl₂, CHCl₃, ClCH₂CH₂Cl, N(C₁-C₆ alkyl)₃, N(benzyl)₃, HO(C₁-C₆ alkyl), acetone methylethylketone, methylbutylketone, and mixtures thereof.

In a more preferred embodiment of the process for preparing the compounds of formula 1, the solvent is selected from the group consisting of toluene, dimethylformamide, dimethylacetamide, dioxane, tetrahydrofuran, acetonitrile, N-methylpyrrolidinone, dimethoxyethane, ClCH₂CH₂Cl, N(C₁-C₆ alkyl)₃, N(benzyl)₃, HO(C₁-C₆ alkyl), acetone, methylethylketone, methylbutylketone, and mixtures thereof.

In an even more preferred embodiment of the process for preparing the compounds of formula 1, the solvent is selected from tetrahydrofuran, dioxane, dimethoxyethane, dimethylformamide, dimethylacetamide, N(C₁-C₆ alkyl)₃, N(benzyl)₃, HO(C₁-C₆ alkyl), acetone, methylethylketone, methylbutylketone, and mixtures thereof.

In a most preferred embodiment of the process for preparing the compounds of formula 1, the solvent is 2-butanol (sec-butanol), isopropanol, acetone, methylethylketone, triethylamine, or a mixture thereof.

In a preferred embodiment of the process for preparing the compounds of formula 1, the reaction of compounds of formula 2 and 3 is carried out at a temperature ranging from about 25° C. to about 175° C.

In one embodiment of the presently claimed process for preparing the compounds of formula 1 wherein X is chlorine, the reaction of compounds of formula 2 and 3 is carried out in the presence of a catalyst, ligand, base, and solvent mixture comprised of one of the following:
(i) said catalyst is Pd₂(dba)₃ or Pd(OAc)₂, said ligand is 2-methyl-2′-(dicyclohexylphosphino)biphenyl, 2-dimethylamino-2′-(dicyclohexylphosphino)biphenyl, and P(R²²)₃, wherein R²² is selected from the group consisting of C₁-C₆ alkyl, 2-methyl-2′-(dicyclohexylphosphino)biphenyl and 2-dimethylamino-2′-(dicyclohexylphosphino)biphenyl, said base is selected from the group consisting of M₂CO₃, M₃PO₄, and MX wherein M is selected from the group consisting of Na, K, Cs, and (R)₄N, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 4 and said solvent is selected from the group consisting of toluene, benzene, xylene, DME, acetone, Dioxane, DMF, DMAC, NMP, and ACN;
(ii) said catalyst is selected from the group consisting of Pd(OAc)₂, PdCl₂, Pd(MeCN)₂Cl₂, Pd(PhCN)₂Cl₂, and PdCl₂(PPh₃)₂, said ligand is Ph₄PX, wherein X is select from the group consisting of Cl, Br, and I, said base is NaOAc or N,N-dimethylglycine, and said solvent is selected from the group consisting DMF, DMAC, water, dioxane, THF, ACN, and NMP;

(iii) said catalyst is selected from the group consisting of trans-di(μ-acetato)-bis[o-(di-o-tolylphosphino)benzyl]dipalladium(II), bis(1,3-dihydro-1,3-dimethyl-2H-imidazol-2-ylidene)diiodo-palladium, and diiodo[methylenebis[3-(2-methyl)-1H-imidazol-1-yl-2(3H)-ylidene]]-palladium, said base is NaOAc, Bu₄NBr, hydrazine, or NaOCHO, and said solvent is selected from the group consisting toluene, benzene, xylene, DME, acetone, dioxane, DMF, DMAC, and NMP; or
(iv) said catalyst is Pd₂(dba)₃, said ligand is 1,3-Bis(2,4,6-trimethylphenyl)imidazolium chloride or 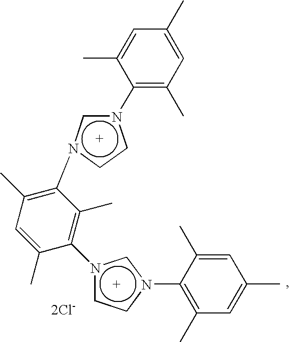
   said base is selected from the group consisting NaOAc, Bu₄NBr, hydrazine, and NaOCHO and said solvent is selected from the group consisting toluene, benzene, xylene, DME, acetone, dioxane, DMF, DMAC, and NMP.

In one embodiment of the presently claimed process for preparing the compounds of formula 4 and 1, R³ is —(CR¹R²)ₜ(4 to 10 membered heterocyclic), wherein t is an integer from 0 to 5, and the foregoing R³ groups are optionally substituted by 1 to 3 R¹⁰ groups; said heterocyclic group is optionally fused to a benzene ring or a C₅-C₈ cycloalkyl group, and the foregoing R³ groups, including any optional fused rings referred to above, are optionally substituted by 1 to 3 R¹⁰ groups.

Another embodiment of the present invention refers to those methods wherein R³ is selected from 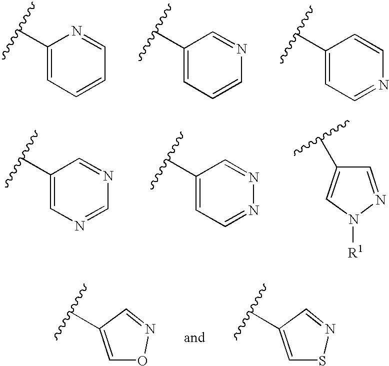
wherein the foregoing R³ groups are optionally substituted by 1 to 3 R¹⁰ groups.

Another embodiment of the present invention refers to those methods wherein R³ is pyridin-3-yl optionally substituted by 1 to 3 R¹⁰ groups.

Another embodiment of the present invention refers to those methods wherein R⁴ and R⁵ are both hydrogens; in another embodiment, R¹³ and R¹⁴ are both hydrogens; in another embodiment, R¹⁵ and R¹⁶ are both hydrogens; and in another embodiment, R⁴, R⁵, R¹³, R¹⁴, R¹⁵ and R¹⁶ are all hydrogens.

Another embodiment of the present invention refers to those methods wherein k is 1; in another preferred embodiment l is 1. In another preferred embodiment, both k and l are 1.

Another embodiment of the present invention refers to those methods wherein R¹⁷ is a t-butyl group. In another preferred embodiment, R¹⁹ and R²⁰ are both OR¹⁸ wherein each R¹⁸ independently is a C₁-C₆ alkyl group; in another preferred embodiment, R¹⁸ is a t-butyl group. In another preferred embodiment, R¹⁹ is —(CR¹⁵R¹⁶)ₗOR¹⁷ and R²⁰ is OR¹⁸ wherein R¹⁵, R¹⁶ R¹⁷, and R¹⁸ are as defined for formula 1.

The present invention also relates to a method for preparing a compound of formula 5 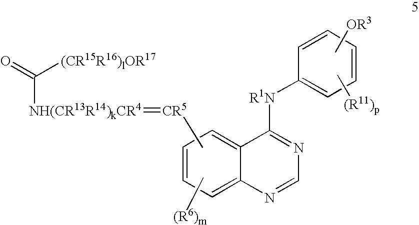
comprising converting a compound of formula 1 in one or more steps to produce the compound of formula 5.

In one embodiment of this process to arrive at compound of formula 5 from the compound of formula 1, the steps comprise:
(a) reacting the compound of formula 1 with an acid to form a compound of formula 4 or a salt thereof 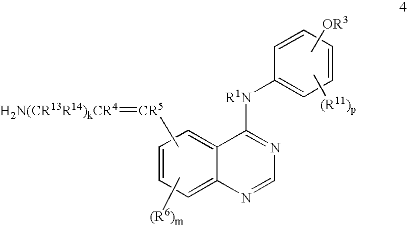
   and
(b) reacting the compound of formula 4 or its salt with ClC(O)(CR¹⁵R¹⁶)ₗOR¹⁷, or a reactive equivalent thereof wherein R¹⁵, R¹⁶, R¹⁷ and l are as defined for formula 1 in the presence of a base to form the compound of formula 5. Reactive equivalents of acid chlorides include without limitation, carboxylic acids, acid anhydrides and acid imidazoles. In one preferred embodiment, a reactive equivalent of the acid chloride ClC(O)(CR¹⁵R¹⁶)ₗOR¹⁷ is an acid imidazole represented by the formula 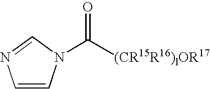
   wherein R¹⁵, R¹⁶, R¹⁷ and l are as defined for formula 1. In one embodiment, a reactive equivalent of the acid chloride ClC(O)(CR¹⁵R¹⁶)ₗOR¹⁷ is an acid anhydride represented by the formula [R¹⁷O(CR¹⁵R¹⁶)ₗC(O)]₂O.

The acid used to react with the compound of formula 1 to form compound 4 in step (a) may be any acid, including mineral acids, carboxylic acids and organic sulfonic acids.

The base used in step (b) can be at least one compound selected from the group consisting of an aqueous hydroxide of an alkali or alkaline earth metal, a carbonate, phosphate or hydrogen phosphate of an alkaline earth metal, an tertiary amine and DABCO. Preferably the base is at least one compound selected from the group consisting of NaOH, KOH, Et₃N, Me₂NEt, iPr₂NEt, K₂CO₃, Na₃PO₄, Na₂HPO₄, DABCO, and 1,8-(dimethylamino)naphthalene.

In another embodiment of this process to arrive at compound of formula 5 from the compound of formula 1, the step comprises reacting the compound of formula 1 with an acid in one step to produce the compound of formula 5. The acid can be any acid, including mineral acids, carboxylic acids and organic sulfonic acids.

Examples of compounds of formula 5 that can be prepared from the compounds of formula 1 as disclosed in the aforementioned process include the following compounds:
E-2-Methoxy-N-(3-{4-[3-methyl-4-(6-methyl-pyridin-3-yloxy)-phenylamino]-quinazolin-6-yl}-allyl)-acetamide;
E-N-(3-{4-[3-Chloro-4-(6-methyl-pyridin-3-yloxy)-phenylamino]-quinazolin-6-yl}-allyl)-2-methoxy-acetamide;
E-N-(3-{4-[3-Chloro-4-(6-methyl-pyridin-3-yloxy)-phenylamino]-quinazolin-6-yl}-allyl)-acetamide;
E-2-Ethoxy-N-(3-{4-[3-methyl-4-(6-methyl-pyridin-3-yloxy)-phenylamino]-quinazolin-6-yl}-allyl)-acetamide;
E-N-(3-{4-[3-Methyl-4-(6-methyl-pyridin-3-yloxy)-phenylamino]-quinazolin-6-yl}-allyl)-methanesulfonamide;
   and the pharmaceutically acceptable salts, prodrugs and solvates of the foregoing compounds.

The present invention also relates to a process for preparing a compound represented by the formula 3a 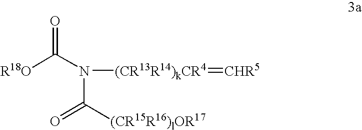
wherein R⁴ and R⁵ are independently selected from hydrogen and C₁-C₆ alkyl; each R¹³, R¹⁴, R¹⁵ and R¹⁶ is independently selected from hydrogen, C₁-C₆ alkyl and CH₂OH, R¹⁷ and R¹⁸ are independently C₁-C₆ alkyl, and k and l are independently an integer from 1 to 3, comprising the steps of:
(a) reacting an amine represented by the formula H₂N—(CR¹³R¹⁴)ₖCR⁴═CHR⁵ wherein R⁴, R⁵, R¹³, R¹⁴ and k are as defined for formula 3a, with a compound represented by the formula R¹⁷O(R¹⁶R¹⁵C)ₗC(O)X where X is a halide, or a reactive equivalent of the formula R¹⁷O(R¹⁶R¹⁵C)ₗC(O)X to form a compound represented by the formula 6 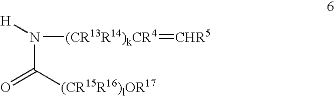
   wherein R⁴, R⁵, R¹³, R¹⁴, R¹⁵, R¹⁶, R¹⁷, k and l are as defined for formula 3a above;
and (b) reacting the compound represented by the formula 6 with a compound of formula (R¹⁸OC(O))₂O or a reactive equivalent thereof optionally in the presence of a basic catalyst to form the compound represented by the formula 3a.

Reactive equivalents of acid chlorides include without limitation, carboxylic acids, acid anhydrides and acid imidazoles.

Preferably the halide X is a bromide, or an iodide. In an especially preferred embodiment of the process for preparing the compounds of formula 3a, the basic catalyst is dimethylaminopyridine (DMAP). In a preferred embodiment of the process for preparing the compounds of formula 3a, R⁴ and R⁵ are both hydrogen; in another preferred embodiment, R¹³, R¹⁴, R¹⁵ and R¹⁶ are all hydrogens; and in another preferred embodiment, R⁴, R⁵, R¹³, R¹⁴, R¹⁵, and R¹⁶ are all hydrogens. In another preferred embodiment, k and l are both 1; and in another preferred embodiment, R¹⁷ is methyl and R¹⁸ is t-butyl.

The present invention also relates to a compound represented by the formula 3a set forth above.

In one preferred embodiment of the compounds of formula 3a, R⁴ and R⁵ are both hydrogens. In another preferred embodiment of the compounds of formula 3a, R¹³, R¹⁴, R¹⁵ and R¹⁶ are all hydrogens. In another preferred embodiment of the compounds of formula 3a, k and l are both 1. In another preferred embodiment of the compounds of formula 3a, R¹⁷ is methyl and R¹⁸ is t-butyl.

The compound of formula 3a is useful as a starting material for the preparation of the compounds of formula 1 and 5.

Compounds of formula 5 are capable of inhibiting abnormal cell growth, such as cancer, in mammals and are selective inhibitors of selective receptor tyrosine kinases.

The term “halo”, as used herein, unless otherwise indicated, includes fluoro, chloro, bromo or iodo. Preferred halo groups are fluoro and chloro.

The term “alkyl”, as used herein, unless otherwise indicated, includes saturated monovalent hydrocarbon radicals having straight, cyclic (including mono- or multi-cyclic moieties) or branched moieties. It is understood that for said alkyl group to include cyclic moieties it must contain at least three carbon atoms.

The term “cycloalkyl”, as used herein, unless otherwise indicated, includes saturated monovalent hydrocarbon radicals having cyclic (including mono- or multi-cyclic) moieties.

The term “alkenyl”, as used herein, unless otherwise indicated, includes alkyl groups, as defined above, having at least one carbon-carbon double bond.

The term “alkynyl”, as used herein, unless otherwise indicated, includes alkyl groups, as defined above, having at least one carbon-carbon triple bond.

The term “aryl” or “Ar”, as used herein, unless otherwise indicated, includes an organic radical derived from an aromatic hydrocarbon by removal of one hydrogen, such as phenyl or naphthyl. “Aryl” or “Ar” are optionally substituted with 1 to 4 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5, wherein t, R¹, R², R⁶, and R⁷ are as defined for formula 1.

The term “alkoxy”, as used herein, unless otherwise indicated, includes —O-alkyl groups wherein alkyl is as defined above.

The term “4 to 10 membered heterocyclic”, as used herein, unless otherwise indicated, includes aromatic and non-aromatic heterocyclic groups containing one or more heteroatoms each selected from O, S and N, wherein each heterocyclic group has from 4 to 10 atoms in its ring system. Non-aromatic heterocyclic groups include groups having only 4 atoms in their ring system, but aromatic heterocyclic groups must have at least 5 atoms in their ring system. The heterocyclic groups include benzo-fused ring systems and ring systems substituted with one or more oxo moieties. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the compounds listed above, may be C-attached or N-attached where such is possible. For instance, a group derived from pyrrole may be pyrrol-1-yl (N-attached) or pyrrol-3-yl (C-attached).

The term “Me” means methyl, “Et” means ethyl, and “Ac” means acetyl.

The term “DME”, as used herein, unless otherwise indicated, means dimethoxyethane.

The term “DMF”, as used herein, unless otherwise indicated, means dimethylformamide.

The term “DMAC”, as used herein, unless otherwise indicated, means dimethylacetamide.

The term “ACN”, as used herein, unless otherwise indicated, means acetonitrile.

The term “NMP”, as used herein, unless otherwise indicated, means N-methylpyrrolidinone.

The term “DMSO”, as used herein, unless otherwise indicated, means dimethylsulfoxide.

The term “BINAP” (abbreviation for 2,2′-Bis(diphenylphosphino)-1,1′-binaphthyl), as used herein, unless otherwise indicated, is represented by the following formula:

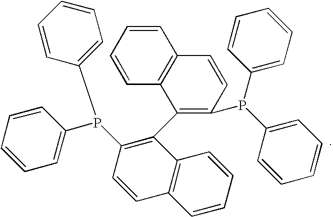

The term “DABCO”, as used herein, unless otherwise indicated, means 1,4-diazabicyclo[2.2.2]octane.

The term “DBA”, as used herein, unless otherwise indicated, means dibenzanthracene.

The term “dppe”, as used herein, unless otherwise indicated, means Ph₂P(CH₂)₂PPh₂.

The term “dppp”, as used herein, unless otherwise indicated, means Ph₂P(CH₂)₃PPh₂.

The term “dppb”, as used herein, unless otherwise indicated, means Ph₂P(CH₂)₄PPh₂.

The term “dippb”, as used herein, unless otherwise indicated, means iPr₂P(CH₂)₄PiPr₂.

The term “dppf”, as used herein, unless otherwise indicated, is represented by the following formula:

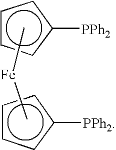

The term “Otfa”, as used herein, unless otherwise indicated, means O₂CCF₃.

The term “R”, as used herein, unless otherwise indicated, means it is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, wherein R¹ and R² are as defined above for formula 1.

The compound trans-di(μ-acetato)-bis[o-(di-o-tolylphosphino)benzyl]dipalladium(II) is represented by the formula

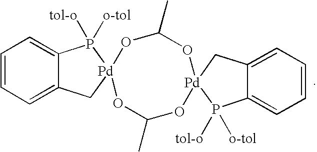

The compound bis(1,3-dihydro-1,3-dimethyl-2H-imidazol-2-ylidene)diiodo-palladium is represented by the formula

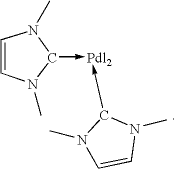

The compound diiodo[methylenebis[3-(2-methyl)-1H-imidazol-1-yl-2(3H)-ylidene]]-palladium is represented by the formula

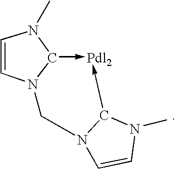

The compound 1,3-Bis(2,4,6-trimethylphenyl)imidazolium chloride is represented by the formula

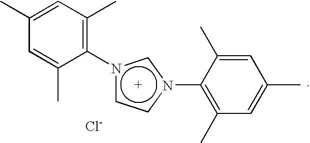

The term “reactive equivalent” of a material means any compound or chemical composition other than the material itself which reacts or behaves like the material itself under the reaction conditions. Thus reactive equivalents of carboxylic acids will include acid-producing derivatives such as anhydrides, acyl halides, and mixtures thereof unless specifically stated otherwise. One of ordinary skill in the art that will recognize that the phrase “synthetic equivalent” or “synthon” is a synonym for “reactive equivalent” (see, e.g., Warren, Stuart, “Designing Organic Synthesis, A Programmed Introduction to the Synthon Approach”, John Wiley & Sons, New York, 1978, p.8).

The present invention also includes isotopically-labelled compounds, which are identical to those recited in Formula 1, 3a or 5, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

Compounds of the present invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the present invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

Each of the documents referred to in this patent application is incorporated herein by reference in its entirety.

### DETAILED DESCRIPTION OF THE INVENTION

Compounds of the formulae 1 and 5 may be prepared according to the following reaction schemes and discussion. Unless otherwise indicated R¹, R³, R⁴,R⁵,R⁶, R¹¹, R¹³, R¹⁴, R¹⁵, R¹⁶, R¹⁷, R¹⁹, R²⁰, k, l, m and p and structural formulae 1, 4 and 5 in the reaction schemes and discussion that follow are as defined above.

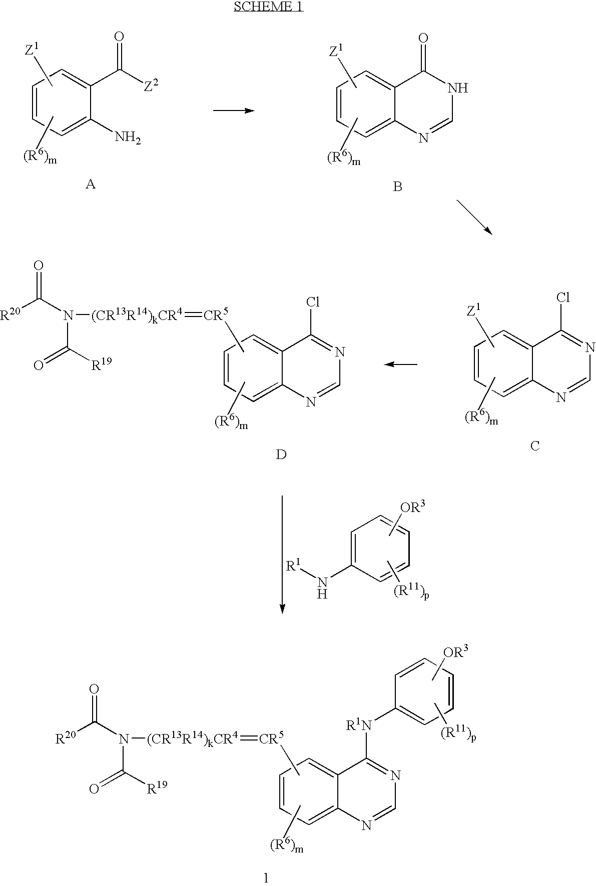

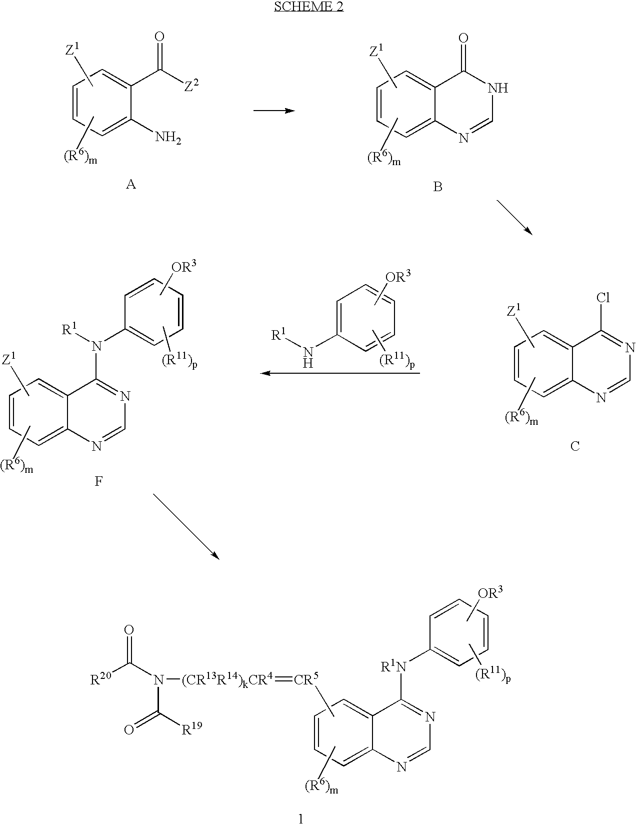

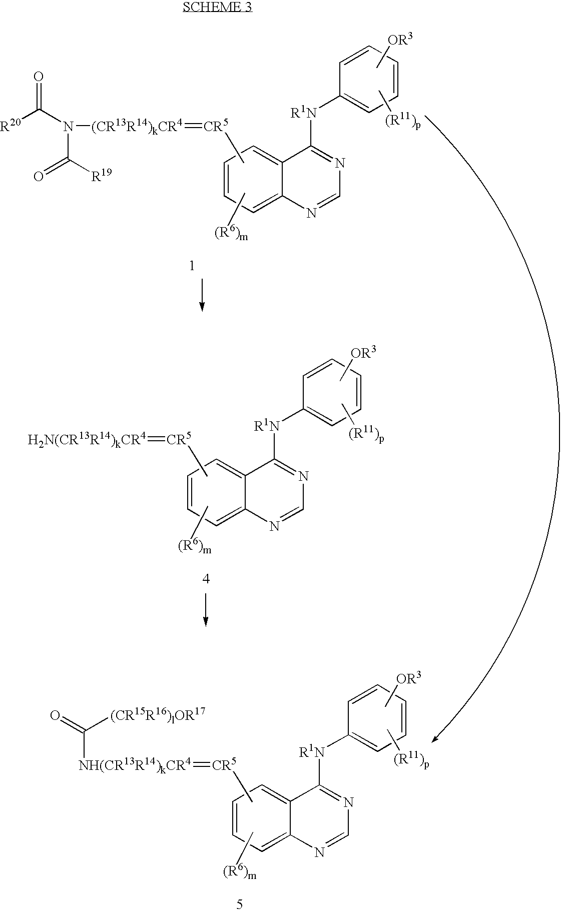

With reference to Scheme 1 above, the compound of formula 1 may be prepared by coupling the compound of formula D, with an amine of formula E, in an anhydrous solvent, in particular a solvent selected from DMF (N,N-dimethylformamide), DME (ethylene glycol dimethyl ether), DCE (dichloroethane) and t-butanol, and phenol, or a mixture of the foregoing solvents, a temperature within the range of about 50-150° C. for a period ranging from 1 hour to 48 hours. The heteroaryloxyanilines of formula E may be prepared by methods known to those skilled in the art, such as, reduction of the corresponding nitro intermediates. Reduction of aromatic nitro groups may be performed by methods outlined in Brown, R. K., Nelson, N. A. J. Org. Chem. 1954, p. 5149; Yuste, R., Saldana, M, Walls, F., Tet. Lett. 1982, 23, 2, p. 147; or in WO 96/09294, referred to above. Appropriate heteroaryloxy nitrobenzene derivatives may be prepared from halo nitrobenzene precursors by nucleophilic displacement of the halide with an appropriate alcohol as described in Dinsmore, C. J. et. al., Bioorg. Med. Chem. Lett., 7, 10, 1997, 1345; Loupy, A. et. al., Synth. Commun., 20, 18, 1990, 2855; or Brunelle, D. J., Tet. Lett., 25, 32, 1984, 3383. Compounds of formula E in which R¹ is a C₁-C₆ alkyl group may be prepared by reductive amination of the parent aniline with R¹CH(O). The compound of formula D may be prepared by treating a compound of formula C, wherein Z¹ is an activating group, such as bromo, iodo, —N₂, or —OTf (which is —OSO₂CF₃), or the precursor of an activating group such as NO₂, NH₂ or OH, with a coupling partner, such as a terminal alkyne, terminal alkene, vinyl halide, vinyl stannane, vinylborane, alkyl borane, or an alkyl or alkenyl zinc reagent. The compound of formula C can be prepared by treating a compound of formula B with a chlorinating reagent such as POCl₃, SOCl₂ or ClC(O)C(O)Cl/DMF in a halogenated solvent at a temperature ranging from about 60° C. to 150° C. for a period ranging from about 2 to 24 hours. Compounds of formula B may be prepared from a compound of formula A wherein Z¹ is as described above and Z² is NH₂, C₁-C₆ alkoxy or OH, according to one or more procedures described in WO 95/19774, referred to above.

The compounds and reactions in Scheme 2 may be prepared using the methods described for Scheme 1, with one change to the reaction scheme. The compound of formula C is treated with the heteroaryloxyanilines of formula E to form the compound formula F prior to the reaction of the Z¹ activating group with a coupling partner as described above in Scheme 1.

Scheme 3 shows that the compound of formula 1 can be converted directly to the compound of formula 5 or through the intermediate compound of formula 4, as disclosed hereinabove.

Methods used to prepare the compound of formula 1 may involve standard techniques. These techniques are known to those skilled in the art and include a) removal of a protecting group by methods outlined in T. W. Greene and P. G. M. Wuts, “Protective Groups in Organic Synthesis”, Second Edition, John Wiley and Sons, New York, 1991; b) displacement of a leaving group (halide, mesylate, tosylate, etc) with a primary or secondary amine, thiol or alcohol to form a secondary or tertiary amine, thioether or ether, respectively; c) treatment of phenyl (or substituted phenyl) carbamates with primary of secondary amines to form the corresponding ureas as in Thavonekham, B et. al. Synthesis (1997), 10, p1189; d) reduction of propargyl or homopropargyl alcohols or N-BOC protected primary amines to the corresponding E-allylic or E-homoallylic derivatives by treatment with sodium bis(2-methoxyethoxy)aluminum hydride (Red-Al) as in Denmark, S. E.; Jones, T. K. J. Org. Chem. (1982) 47, 4595-4597 or van Benthem, R. A. T. M.; Michels, J. J.; Speckamp, W. N. Synlett (1994), 368-370; e) reduction of alkynes to the corresponding Z-alkene derivatives by treatment hydrogen gas and a Pd catalyst as in Tomassy, B. et. al. Synth. Commun. (1998), 28, p1201 f) treatment of primary and secondary amines with an isocyanate, acid chloride (or other activated carboxylic acid derivative), alkyl/aryl chloroformate or sulfonyl chloride to provide the corresponding urea, amide, carbamate or sulfonamide; g) reductive amination of a primary or secondary amine using R¹CH(O); and h) treatment of alcohols with an isocyanate, acid chloride (or other activated carboxylic acid derivative), alkyl/aryl chloroformate or sulfonyl chloride to provide the corresponding carbamate, ester, carbonate or sulfonic acid ester.

The presently claimed process of preparing the compound of formula 1 by reacting the compound of formula 2 with the compound of formula 3 as set forth above is a Heck reaction. The following review articles, hereby incorporated by reference, identify reagents that may be employed in the Heck reaction to prepared the compounds of the present invention: (a) Heck, R. F. in Comprehensive Organic Synthesis; Trost, B. M., Ed.; Pergamon: New York, 1991; Vol. 4, Chapter 4.3; (b) Bräse, S.; deMeijere, A. in Metal-catalyzed Cross-coupling Reactions; Deiderich, F.; Stang, P. J., Eds.; Wiley: New York, 1998, Chapter 3; (c) Cabri, W.; Candiani, I. Acc. Chem. Res. 1995, 28, 2-7; and (d) deMeijere, A.; Meyer; F. E. Angew. Chem. Int. Ed. Engl. 1994, 33, 2379-2411.

In one preferred embodiment of the process of the present invention the Heck reactions employ aryl chlorides. The following articles disclose the use of aryl chlorides in the Heck reaction, which are hereby incorporated by reference: (a) Riermeier, T. H.; Zapf, A.; Beller, M. Top. Catal. 1997, 4, 301-309; (b) Littke, A. F.; Fu, G. C. J. Org. Chem. 1999, 64, 10-11; (c) Reetz, M. T.; Lohmer, G.; Schwickardi, R. Angew. Chem. Int. Ed. 1998, 37, 481-483; (d) Beller, M.; Zapf, A. Synlett 1998, 792-793; (e) Ben-David, Y.; Portnoy, M.; Gozin, M.; Milstein, D. Organometallics 1992, 11, 1995-1996; (f) Portnoy, M.; Ben-David, Y.; Milstein, D. Organometallics 1993, 12, 4734-4735; (g) Portnoy, M.; Ben-David, Y.; Rousso, I.; Milstein, D. Organometallics 1994, 13, 3465-3479; (h) Herrmann, W. A.; Brossmer, C.; Öfele, K.; Reisinger, C.-P.; Priermeier, T.; Beller, M.; Fischer H. Angew. Chem. Int. Ed. Engl. 1995, 34, 1844-1848; (i) Herrmann, W. A.; Elison, M.; Fischer J.; Köcher, C.; Artus, G. R. J. Angew. Chem. Int. Ed. Engl. 1995, 34, 2371-2374; and (j) Herrmann, W. A.; Brossmer, C.; Reisinger, C.-P.; Riermeier, T. H.; Öfele, K.; Beller, M. Chem. Eur. J. 1997, 3, 1357-1364.

The following table lists preferred Pd catalysts, ligands, bases, and solvents from Bräse, S.; deMeijere, A. in Metal-catalyzed Cross-coupling Reactions; Deiderich, F.; Stang, P. J., Eds.; Wiley: New York, 1998; Chapter 3, pages 108-109 for use in the Heck reaction.

| Pd source | Ligand | Base | Solvent |
|---|---|---|---|
| Pd(PPh₃)₄ | PAr₃, preferably | DABCO, proton | toluene, benzene, |
| PdCl₂(PPh₃)₂, or | PPh₃, P(o-Tol)₃, P(o- | sponge, (R)₂NH, | xylene, DMF, DMAC, |
| BnPdCl(PPh₃)₂. | OMePh)₃, P(2- | (R)NH₂ | water, dioxane, THF, |
| Pd(OAc)₂, | Furyl)₃, | (R)₃N, | ACN, NMP, DMSO, |
| Pd(O₂CCF₃)₂, or | BINAP, dppf, dppe, | QX, wherein X is | MeOH, EtOH, iPrOH, |
| Pd(PPh₃)₂(O₂CCF₃)₂. | dppb, or dppp. | F, Cl, or Br, | DME, acetone. |
| Pd (Pd/C, Pd black, | Polymer bound | Q(CO₃) | CH₂Cl₂, CHCl₃, |
| Pd on other solid | phosphines | QH(PO₄) | ClCH₂CH₂Cl, NR₃, |
| supports such as | | Q(OCOR). | preferably NEt₃ or |
| silica, graphite, clay). | | preferably NaOAc. | iPr₂NEt. |
| PdCl₂, Pd(MeCN)₂Cl₂, | | | |
| or Pd(PhCN)₂Cl₂. | | | |
| PdCl₂(dppf), | | | |
| Pd(acac)₂, Pd₂(dba)₃, | | | |
| Pd(dppb), Pd₂(dba)₃- | | | |
| or CHCl₃. | | | |
| P(Ar)₃, preferably | | | |
| PPh₃, P(o-Tol)₃, P(o- | | | |
| OMePh)₃, P(2-Furyl)₃, | | | |

In one preferred embodiment when X is Cl the following Table lists the Pd catalyst, ligand, based and solvent, which may be employed for the preparation of the compounds of formula 1 using the Heck reaction.

| Pd source | Ligand | Base | Solvent |
|---|---|---|---|
| Pd₂(dba)₃ or | P(R)₃, preferably | Q(CO₃), preferably | toluene, benzene, |
| Pd(OAc)₂ | P(t-Bu)₃ or P(i-Pr)₃. | Cs₂(CO₃). | xylene, DME, acetone |
| | | | Dioxane, DMF, |
| | | | DMAC, NMP, or ACN. |
| Pd(OAc)₂, PdCl₂, | Ph₄PX, wherein X is | NaOAc or | DME, DMAC, water, |
| Pd(MeCN)₂Cl₂, | Cl, Br, or I. | NN dimethylglycine | dioxane, THF, ACN, |
| Pd(PhCN)₂Cl₂, or | | | or NMP. |
| PdCl₂(PPh₃)₂ | | | |
| Pd(OAc)₂, PdCl₂, | P(OR)₃, wherein R is | Q(OCOR), | DMF, DMAC, water, |
| Pd(MeCN)₂Cl₂, or | Et, iPr, Ph, 2,4-dit- | preferably NaOAc, | dioxane, THF, ACN, |
| Pd(PhCN)₂Cl₂, | BuPh, Ar, | and Q(CO₃), | or NMP. |
| | Or dippb. | preferably Na₂CO₃. | |
| Palladacycle 1 | No Ligand | NaOAc, Bu₄NBr, | DMAc, DMF, or NMP. |
| Catalysts 1-2 | | hydrazine, or | |
| | | NaOCHO. | |
| Pd₂(dba)₃ | Ligand 1 | NaOAc, Bu₄NBr, | DMAc, DMF, or NMP. |
| | | hydrazine, or | |
| | | NaOCHO. | |

Preferably, the palladium catalyst employed in the present invention is a palladium(0) catalyst, more preferably the palladium(0) catalyst is tetrakis(triphenylphosphine)palladium(0) or Pd₂(dba)₃. This may be added to the reaction mixture directly or generated in situ by adding triphenylphosphine and palladium acetate which is converted to palladium(0) species under the reaction conditions.

General synthetic methods which may be referred to for preparing the compounds of the present invention are provided in U.S. Pat. No. 5,747,498 (issued May 5, 1998), U.S. patent application Ser. No. 08/953,078 (filed Oct. 17, 1997), WO 98/02434 (published Jan. 22, 1998), WO 98/02438 (published Jan. 22, 1998), WO 96/40142 (published Dec. 19, 1996), WO 96/09294 (published Mar. 6, 1996), WO 97/03069 (published Jan. 30, 1997), WO 95/19774 (published Jul. 27, 1995) and WO 97/13771 (published Apr. 17, 1997). Additional procedures are referred to in World Patent Application WO 00/44728 (published Aug. 3, 2000) and European patent publication EP 1029853 (published Aug. 23, 2000). The foregoing patents and patent applications are incorporated herein by reference in their entirety. Certain starting materials may be prepared according to methods familiar to those skilled in the art and certain synthetic modifications may be done according to methods familiar to those skilled in the art. A standard procedure for preparing 6-iodoquinazolinone is provided in Stevenson, T. M., Kazmierczak, F., Leonard, N. J., J. Org. Chem. 1986, 51, 5, p. 616. Palladium-catalyzed boronic acid couplings are described in Miyaura, N., Yanagi, T., Suzuki, A. Syn. Comm. 1981,11, 7, p. 513. Palladium catalyzed Heck couplings are described in Heck et. al. Organic Reactions, 1982, 27, 345 or Cabri et. al. in Acc. Chem. Res. 1995, 28, 2. For examples of the palladium catalyzed coupling of terminal alkynes to aryl halides see: Castro et. al. J. Org. Chem. 1963, 28, 3136. or Sonogashira et. al. Synthesis, 1977, 777. Terminal alkyne synthesis may be performed using appropriately substituted/protected aldehydes as described in: Colvin, E. W. J. et. al. Chem. Soc. Perkin Trans. I, 1977, 869; Gilbert, J. C. et. al. J. Org. Chem., 47, 10, 1982; Hauske, J. R. et. al. Tet. Lett., 33, 26, 1992, 3715; Ohira, S. et. al. J. Chem. Soc. Chem. Commun., 9, 1992, 721; Trost, B. M. J. Amer. Chem. Soc., 119, 4, 1997, 698; or Marshall, J. A. et. al. J. Org. Chem., 62, 13, 1997, 4313.

Alternatively terminal alkynes may be prepared by a two step procedure. First, the addition of the lithium anion of TMS (trimethylsilyl) acetylene to an appropriately substituted/protected aldehyde as in: Nakatani, K. et. al. Tetrahedron, 49, 9, 1993, 1901. Subsequent deprotection by base may then be used to isolate the intermediate terminal alkyne as in Malacria, M.; Tetrahedron, 33, 1977, 2813; or White, J. D. et. al. Tet. Lett., 31, 1, 1990, 59.

Starting materials, the synthesis of which is not specifically described above, are either commercially available or can be prepared using methods well known to those of skill in the art.

In each of the reactions discussed or illustrated in the Schemes, pressure is not critical unless otherwise indicated. Pressures from about 0.5 atmospheres to about 5 atmospheres are generally acceptable, and ambient pressure, i.e., about 1 atmosphere, is preferred as a matter of convenience.

The examples and preparations provided below further illustrate and exemplify the compounds of the present invention, methods of preparing such compounds, and the methods of the present invention. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Where HPLC chromatography is referred to in the preparations and examples below, the general conditions used, unless otherwise indicated, are as follows. The column used is a ZORBAX RXC18 column (manufactured by Hewlett Packard) of 150 mm distance and 4.6 mm interior diameter. The samples are run on a Hewlett Packard-1100 system. A gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 mL/minute.

The present invention is illustrated by the following Examples. It will be understood, however, that the invention is not limited by the specific details of the following Examples.

### EXAMPLE 1

Preparation of di-tert-butyl allylimino dicarboxylate

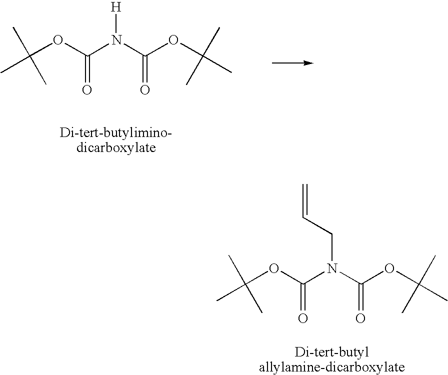

To an appropriate round bottom flask with 150 mL of 2-methyltetrahydrofuran (“2-methylTHF”) was added di-tert-butylimino-dicarboxylate (25.0 g, 115 mmol), allyl bromide (16.7 g, 12.0 mL, 138 mmol), and terabutylammonium bromide (0.520 g 1.61 mmol). In a second flask a sodium hydroxide solution is prepared by adding sodium hydroxide pellets (23.0 g, 576 mmol) to 100.0 mL of process water at 0°-5° C. At room temperature the solution of sodium hydroxide is added to the reaction. The reaction is heated 40°-50° C. After 1 hour HPLC (GTP 6354.01 Armor C-18 5 uM 150×4.6 cm, 20 mM K2HPO4-pH 7), showed total consumption of di-tert-butylimino-dicarboxylate. Separated the layers and the 2-methylTHF layer is washed with process water. The organic layer is displaced with isopropanol to a KF of 0.1-0.2% and used as a solution in isopropanol for the next step. HPLC Method on HP1100 using GTP 6354.01 indicated a main product band at 26.4 minutes with area percent of 96.0%. The yield was 95 to 98%.

¹H NMR (400 MHz; CDCl₃): δ 5.78-5.86 (m, 1H), 5.08-5.17 (m, 2H), 4.16 (d,. J=5.6 Hz, 2H), 1.48 (s, 18H).

### EXAMPLE 2

### Preparation of Allyl-methoxyacetyl-carbamic acid tert-butyl ester

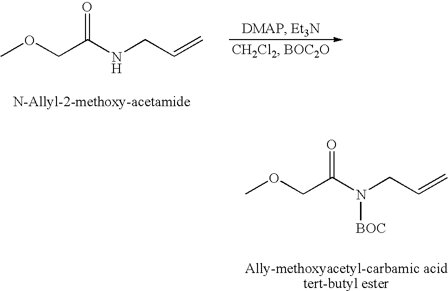

N-Allyl-2-methoxy-acetamide (6.95 g, 54 mmol) was dissolved in a solution of dry CH₂Cl₂ (100 ml). 4-(Dimethylamine)pyridine (54 mmol, 6.6 g) and Et₃N (5.5 g, 54 mmol) were added to the solution. The solution was cooled to 0° C. and BOC₂O (108 mmol, 23.6 g) was added dropwise. The solution was allowed to warm to room temperature and was stirred overnight. The reaction mixture was diluted 100 mL H₂O, and extracted with CH₂Cl₂ (3×50 mL). The combined organic solvents were removed in vacuo to give an oil. This material was then chromatographed on silica gel eluting with 10-20% EtOAc/hexane to give 6.6 g (54%) of title compound as a colorless oil.

¹H NMR (300 MHz, CDCl₃): δ 5.60-5.65 (m, 1H), 4.96-5.02 (m, 2H), 4.38 (s, 2H), 4.15 (d, J=4.5 Hz, 2H), 3.30 (s, 3H), 1.37 (s, 9H).

### EXAMPLE3

### Preparation of bis hydrochloride salt of [6-(3-Amino-propenyl)-quinazolin-4-yl]-[3-methyl-4-(6-methyl-pyridin-3-yloxy)-phenyl]-amine (Procedure A)

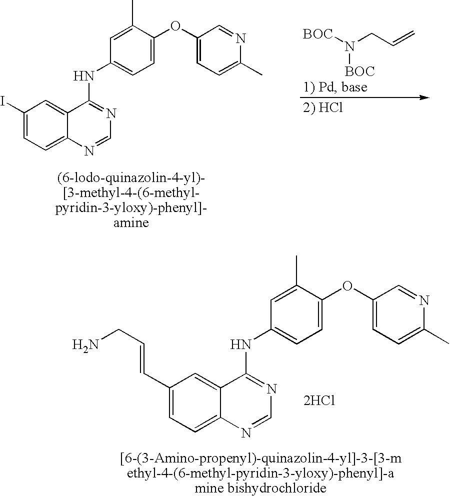

A 100 mL RB flask was charged with (6-Iodo-quinazolin-4-yl)-[3-methyl-4-(6-methyl-pyridin-3-yloxy)-phenyl]-amine hydrochloride (5 g, 10 mmol, 1 eq), Di-tert-butyl allylimino-dicarboxylate (6 g, 23 mmol, 2.3 eq), PPh₃ (265 mg), Pd(OAc)₂ (115 mg, 0.05 eq), NaOAc (3.28 g, 40 mmol, 4 eq) and 75 mL DMF. The resulting homogeneous mixture was heated at 100° C. under N₂ for 6 h, cooled to room temperature, diluted with 100 mL H₂O and extracted with 50 mL EtOAc . The organic solvents were removed in vacuo to give a crude dark brown residue. This material was then dissolved in 50 mL THF. To the THF solution cooled with water bath was added 40 ml concentrated HCl slowly. The resulting mixture was stirred at room temperature for 4 h (the title compound precipitated slowly after about 15 min). The title compound as a light yellow salt was filtered and washed with plenty of THF and vacuum dried. The weight of the product obtained was 3.5 g (80% yield).

¹H NMR (300 MHz, D₂O): δ 8.53 (s, 1H), 8.35 (d, J=1.8 Hz, 1H), 8.22 (d, J=2.4 Hz, 1H), 8.12 (dd, J=9 Hz, 1.5 Hz, 1H), 7.99 (dd, J=9 Hz, 2.7 Hz, 1H), 7.69-7.74 (m,2H), 7.48 (d, J=2.7 Hz, 1H), 7.38 (dd, J=8.7 Hz, 2.4 Hz, 1H), 7.16(d, J=8.7 Hz, 1H), 6.9 (d, J=16.2 Hz, 1H), 6.5 (dt, J=16.2 Hz, 6.6 Hz, 1H), 2.61 (s, 3H), 2.14 (s, 3H).

The title compound was isolated and characterized by HPLC/MS as follows:

HPLC/MS CONDITIONS

| | |
|---|---|
| Instrument: | Hewlett-Packard 1100 series HPLC/MS |
| Column: | Armor C-18, 5 uM, 150 × 4.6 |
| Mobile Phase: | 20 mM K₂HPO₄, pH 7.0, ACN, MeOH + gradient |
| Flow rate: | 1 mL/min |
| Detection: | UV 210 nm |

The title compound had a retention time under the above conditions of 5.54 minutes and a M+1 peak in MS of 398.

### EXAMPLE 4

### Preparation of bis hydrochloride salt of [6-(3-Amino-propenyl)-quinazolin-4-yl]-[3-methyl-4-(6-methyl-pyridin-3-yloxy)-phenyl]-amine (Procedure B)

A 25 mL RB flask was charged with (6-Iodo-quinazolin-4-yl)-[3-methyl-4-(6-methyl-pyridin-3-yloxy)-phenyl]-amine hydrochloride (0.25 g, 0.5 mmol, 1 eq), Di-tert-butyl allylimino-dicarboxylate (0.257 g, 1 mmol, 2 eq), Pd₂(dba)₃ (23 mg), Et₃N (0.505 g, 5 mmol, 10 eq) and 9 mL 2-propanol. The resulting homogeneous mixture was heated at 80° C. under N₂ for 4 h, cooled to room temperature and filtered. The organic solvents were removed in vacuo to give a crude dark brown residue. This material was then dissolved in 5 mL THF. To the THF solution cooled with water bath was added 2 ml conc. HCl slowly. The resulting mixture was stirred at room temperature for 4 h (the title compound precipitated slowly after about 15 min). The title compound as a light yellow salt was filtered and washed with plenty of THF and vacuum dried.

The yield, purity and analytical data match those of the product made by procedure A (Example 3) above.

### EXAMPLE 5

### Preparation of bis hydrochloride salt of [6-(3-Amino-propenyl)-quinazolin-4-yl-]-[3-methyl-4-(6-methyl-pyridin-3-yloxy)-phenyl]-amine (Procedure C)

A RB flask was charged with (6-Iodo-quinazolin-4-yl)-[3-methyl-4-(6-methyl-pyridin-3-yloxy)-phenyl]-amine hydrochloride (50 g, 100 mmol, 1 eq), Di-tert-butyl allylimino-dicarboxylate (28 g, 109 mmol, 1.1 eq), 5% Pd/C (2.1 g, type CP-87, 50% wet), 35 ml triethylamine, and 400 mL 2-butanol. The resulting homogeneous mixture was heated at reflux under N₂ for 48 h, cooled to room temperature, and filtered over celite. To the filtrate was added 40.1 ml concentrated HCl slowly (495 mmol, 5 eq.). The resulting mixture was stirred at 45 C for 24 h (the title compound precipitated slowly after about 15 min). The title compound as a light yellow salt was filtered and washed with plenty of s-butanol and vacuum dried. The weight of the product obtained was 50 g (107% yield, high in water content and HCl).

¹H NMR (300 MHz, D₂O): δ 8.53 (s, 1H), 8.35 (d, J=1.8 Hz, 1H), 8.22 (d, J=2.4 Hz, 1H), 8.12 (dd, J=9 Hz, 1.5 Hz, 1H), 7.99 (dd, J=9 Hz, 2.7 Hz, 1H), 7.69-7.74 (m,2H), 7.48 (d, J=2.7 Hz, 1H), 7.38 (dd, J=8.7 Hz, 2.4 Hz, 1H), 7.16(d, J=8.7 Hz, 1H), 6.9 (d, J=16.2 Hz, 1H), 6.5 (dt, J=16.2 Hz, 6.6 Hz, 1H), 2.61 (s, 3H), 2.14 (s, 3H).

The title compound was isolated and characterized by HPLC/MS as follows:

HPLC/MS CONDITIONS

| | |
|---|---|
| Instrument: | Hewlett-Packard 1100 series HPLC/MS |
| Column: | Armor C-18, 5 uM, 150 × 4.6 |
| Mobile Phase: | 20 mM K₂HPO₄, pH 7.0, ACN, MeOH + gradient |
| Flow rate: | 1 mL/min |
| Detection: | UV 210 nm |

The title compound had a retention time under the above conditions of 5.54 minutes and a M+1 peak in MS of 398.

### EXAMPLE 6

### Preparation of 2-Methoxy-N-(3-{4-[3-methyl-4-(6-methyl-pyridin-3-yloxy)-phenylamino]quinazolin-6-yl}-allyl)-acetamide

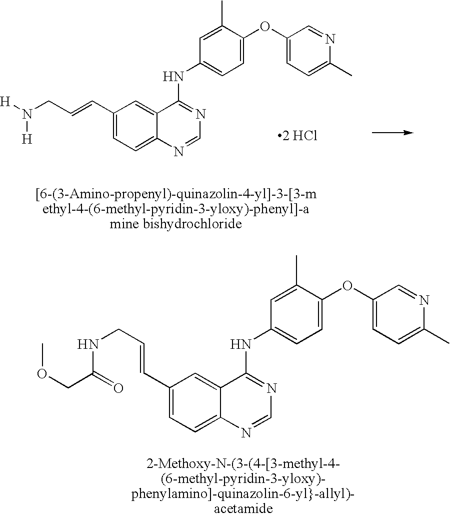

To a stirring solution of [6-(3-amino-propenyl)-quinazolin-4-yl]-[3-methyl-4-(6-methyl-pyridin-3-yloxy)-phenyl]-amine bis hydrochloride (1.0 g, 2.12 mmol) in 10.0 ml of 2-methyltetrahydrofuran was added 10.0 ml of 1N sodium hydroxide solution. Thereafter was added the methoxy acetylchloride (0.254 g, 2.34 mmol). After 1 hour the reaction was deemed complete by HPLC. Reaction was washed with process water. Displaced the 2-methyltetrahydrofuran with ethyl acetate. Off white solid was filtered off to give a 90-94% yield.

¹H NMR (300 MHz, D₂O): δ 8.46 (s, 1H), 8.34 (s, 1H), 8.11 (s, 1H), 7.97 (d, J=7.2 Hz, 1H), 7.70 (d, J=9.2 Hz, 1 H), 7.68 (s, 1H), 7.60 (d, J=6.4 Hz, 1H), 7.27 (dd, 2H), 6.98 (d, J=8.0 Hz, 1H), 6.73 (d, J=16 Hz, 1H), 6.49 (dt, J=16 Hz, 1H), 4.09 (d, J=4.8 Hz, 2H),3.95 (s, 2H), 3.45 (s, 3H), 2.49 (s, 3H), 2.25 (s, 3H).

1. A method for preparing a compound of formula 1 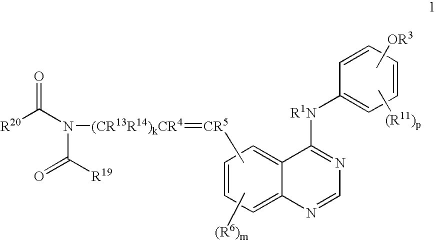
acceptable salts, and solvates thereof, wherein:
k is an integer from 1 to 3;
m is an integer from 0 to 3;
p is an integer from 0 to 4;
R¹, R², R⁴, and R⁵ are each independently selected from H and C₁-C₆ alkyl;
R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond when t is an integer between 2 and 5, and the foregoing R³ group, including any optional fused ring referred to above, is optionally substituted by 1 to 5 R¹⁰ groups;
each R⁶ is independently selected from halo, hydroxy, —NR¹R², C₁-C₆ alkyl, trifluoromethyl, C₁-C₆ alkoxy, trifluoromethoxy, —NR⁷C(O)R¹, —C(O)NR⁷R⁹, —SO₂NR⁷R⁹, —NR⁷C(O)NR⁹R¹, and —NR⁷C(O)OR⁹;
each R⁷, R⁸ and R⁹ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁷, R⁸ and R⁹ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;
or each R⁷ and R⁹, or R⁸ and R⁹, when attached to a nitrogen atom, can be taken together to form a 4 to 10 membered heterocyclic ring which may include 1 to 3 additional hetero moieties, in addition to the nitrogen to which said R⁷, R⁸, and R⁹ are attached, selected from N, N(R¹), O, and S, provided two O atoms, two S atoms or an O and S atom are not attached directly to each other;
each R¹⁰ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁷, —C(O)OR⁷, —OC(O)R⁷, —NR⁷C(O)R⁹, —NR⁷SO₂NR⁹R¹, —NR⁷C(O)NR¹R⁹, —NR⁷C(O)OR⁹, —C(O)NR⁷R⁹, —NR⁷R⁹, —NR⁷OR⁹, —SO₂NR⁷R⁹, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is and interger from 0 to 2, q and t are each independently an integer from 0 to 5, 1 or 2 ring carbon atoms of the heterocyclic moieties of the foregoing R¹⁰ groups are optionally substituted with an oxo (═O) moiety, and the alkyl, alkenyl, alkynyl, aryl and heterocyclic moieties of the foregoing R¹⁰ groups are optionally substituted with 1 to 3 substituents independently selected from halo, cyano, nitro, trifluoromethyl, trifluoromethoxy, azido, —OR⁷, —C(O)R⁷, —C(O)OR⁷, —OC(O)R⁷, —NR⁷C(O)R⁹, —C(O)NR⁷R⁹, —NR⁷R⁹, —NR¹ OR⁹, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
each R¹¹ is independently selected from halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁷, —C(O)OR⁷, —OC(O)R⁷, —NR⁷C(O)R⁹, —NR⁷SO₂NR⁹R¹, —NR⁷C(O)NR¹R⁹, —NR⁷C(O)OR⁹, —C(O)NR⁷R⁹, —NR⁷R⁹, —NR⁷OR⁹, —SO₂NR⁷R⁹, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is an integer from 0 to 2, q and t are each independently an integer from 0 to 5, 1 or 2 ring carbon atoms of the heterocyclic moieties of the foregoing R¹⁰ groups are optionally substituted with an oxo (═O) moiety, and the alkyl, alkenyl, alkynyl, aryl and heterocyclic moieties of the foregoing R¹⁰ groups are optionally substituted with 1 to 3 substituents independently selected from halo, cyano, nitro, trifluoromethyl, trifluoromethoxy, azido, —OR⁷, —C(O)R⁷, —C(O)OR⁷, —OC(O)R⁷, —NR⁷C(O)R⁹, —C(O)NR⁷R⁹, —NR⁷R⁹, —NR⁷OR⁹, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
each R¹³ and R¹⁴ are independently selected from H, C₁-C₆ alkyl, and —CH₂OH;
R¹⁹ and R²⁰ are independently selected from the group consisting of —(CR¹⁵R¹⁶)ₗOR¹⁷ and OR¹⁸ wherein each R¹⁵ and R¹⁶ is independently selected from H, C₁-C₆ alkyl, and —CH₂OH, l is an integer from 1 to 3, R¹⁷ is C₁-C₆ alkyl, R¹⁸ independently is C₁-C₆ alkyl provided both R¹⁹ and R²⁰ are not simultaneously —(CR¹⁵R¹⁶)ₗOR¹⁷;
wherein each carbon not bound to a N or O atom, or to S(O)ⱼ, wherein j is an integer from 0 to 2, is optionally substituted with R¹², wherein R¹² is R⁷, —OR⁷, —OC(O)R⁷, —OC(O)NR⁷R⁹, —OCO₂R⁷, —S(O)ⱼR⁷, —S(O)ⱼNR⁷R⁹, —NR⁷R⁹, —NR⁷C(O)R⁹, —NR⁷SO₂R⁹, —NR⁷C(O)NR⁸R⁹, —NR⁷SO₂NR⁸R⁹, —NR⁷CO₂R⁹,CN, —C(O)R⁷, or halo, wherein j is an integer from 0 to 2; and wherein any of the above-mentioned substituents comprising a CH₃ (methyl), CH₂ (methylene), or CH (methine) group, which is not attached to a halogen, SO or SO₂ group or to a N, O or S atom, is optionally substituted with a group selected from hydroxy, halo, C₁-C₄ alkyl, C₁-C₄ alkoxy and —NR¹R²; which comprises reacting a compound of formula 2 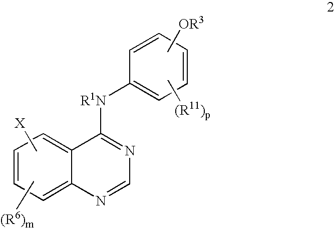
wherein X is a halide and R¹, R³, R⁶, R¹¹, m and p are as defined for formula 1 above, with a compound of formula 3 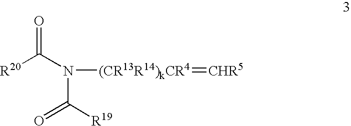
wherein R⁴, R⁵, R¹³, R¹⁴, R¹⁹, R²⁰, and k are as defined for formula 1 above, in the presence of a catalyst, a base, and an optional ligand.

2. A method for preparing a compound of formula 1 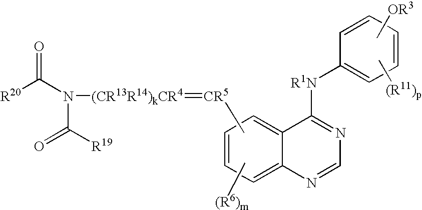
pharmaceutically acceptable salts, solvates and prodrugs thereof, wherein:
m is an integer from 0 to 3;
p is an integer from 0 to 4;
each R¹, R², R⁴, and R⁵ is independently selected from H and C₁-C₆ alkyl;
R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond when t is an integer between 2 and 5, and the foregoing R³ groups, including any optional fused rings referred to above, are optionally substituted by 1 to 5 R¹⁰ groups;
each k and l independently is an integer from 1 to 3;
each R⁶ is independently selected from halo, hydroxy, —NR¹R², C₁-C₆ alkyl, trifluoromethyl, C₁-C₆ alkoxy, trifluoromethoxy, —NR⁷C(O)R¹, —C(O)NR⁷R⁹, —SO₂NR⁷R⁹, —NR⁷C(O)NR⁹R¹, and —NR⁷C(O)OR⁹;
each R⁷, R⁸ and R⁹ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁷, R⁸ and R⁹ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;
or R⁷ and R⁹, or R⁸ and R⁹, when attached to a nitrogen atom, can be taken together to form a 4 to 10 membered heterocyclic ring which may include 1 to 3 additional hetero moieties, in addition to the nitrogen to which said R⁷, R⁸, and R⁹ are attached, selected from N, N(R¹), O, and S, provided two O atoms, two S atoms or an O and S atom are not attached directly to each other;
each R¹⁰ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁷, —C(O)OR⁷, —OC(O)R⁷, —NR⁷C(O)R⁹, —NR⁷SO₂NR⁹R¹, —NR⁷C(O)NR¹R⁹, —NR⁷C(O)OR⁹, —C(O)NR⁷R⁹, —NR⁷R⁹, —NR⁷OR⁹, —SO₂NR⁷R⁹, —S(O)ⱼ(C₁-C₆alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is an integer from 0 to 2, q and t are each independently an integer from 0 to 5, 1 or 2 ring carbon atoms of the heterocyclic moieties of the foregoing R¹⁰ groups are optionally substituted with an oxo (═O) moiety, and the alkyl, alkenyl, alkynyl, aryl and heterocyclic moieties of the foregoing R¹⁰ groups are optionally substituted with 1 to 3 substituents independently selected from halo, cyano, nitro, trifluoromethyl, trifluoromethoxy, azido, —OR⁷, —C(O)R⁷, —C(O)OR⁷, —OC(O)R⁷, —NR⁷C(O)R⁹, —C(O)NR⁷R⁹, —NR⁷R⁹, —NR⁷OR⁹, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
each R¹¹ is independently selected from halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁷, —C(O)OR⁷, —OC(O)R⁷, —NR⁷C(O)R⁹, —NR⁷SO₂NR⁹R¹, —NR⁷C(O)NR¹R⁹, —NR⁷C(O)OR⁹, —C(O)NR⁷R⁹, —NR⁷R⁹, —NR⁷OR⁹, —SO₂NR⁷R⁹, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q} S(O)ⱼ(CR¹R²)ₜ(4 to 10 membered heterocyclic), wherein j is an integer from 0 to 2, q and t are each independently an integer from 0 to 5, 1 or 2 ring carbon atoms of the heterocyclic moieties of the foregoing R¹⁰ groups are optionally substituted with an oxo (═O) moiety, and the alkyl, alkenyl, alkynyl, aryl and heterocyclic moieties of the foregoing R¹⁰ groups are optionally substituted with 1 to 3 substituents independently selected from halo, cyano, nitro, trifluoromethyl, trifluoromethoxy, azido, —OR⁷, —C(O)R⁷, —C(O)OR⁷, —OC(O)R⁷, —NR⁷C(O)R⁹, —C(O)NR⁷R⁹, —NR⁷R⁹, —NR⁷OR⁹, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
each R¹³, R¹⁴, R¹⁵ and R¹⁶ is independently selected from H, C₁-C₆ alkyl, and —CH₂OH;
R¹⁷ is C₁-C₆ alkyl;
wherein each carbon not bound to a N or O atom, or to S(O)ⱼ, wherein j is an integer from 0 to 2, is optionally substituted with R¹², wherein R¹² is R⁷, —OR⁷, —OC(O)R⁷, —OC(O)NR⁷R⁹, —OCO₂R⁷, —S(O)ⱼR⁷, —S(O)ⱼNR⁷R⁹, —NR⁷R⁹, —NR⁷C(O)R⁹, —NR⁷SO₂R⁹, —NR⁷C(O)NR⁸R⁹, —NR⁷SO₂NR⁸R⁹, —NR⁷CO₂R⁹, CN, —C(O)R⁷, or halo, wherein j is an integer from 0 to 2; and wherein any of the above-mentioned substituents comprising a CH₃ (methyl), CH₂ (methylene), or CH (methine) group, which is not attached to a halogen, SO or SO₂ group or to a N, O or S atom, is optionally substituted with a group selected from hydroxy, halo, C₁-C₄ alkyl, C₁-C₄ alkoxy and —NR¹R²; which comprises reacting a compound of formula 7 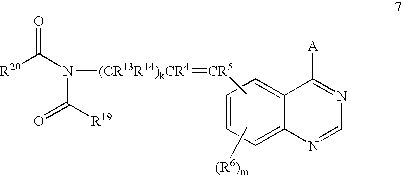
wherein A is Cl or F and R⁴, R⁵, R⁶, R¹³, R¹⁴, R¹⁹, R²⁰, k and m are as defined for formula 1 with a compound of formula 8 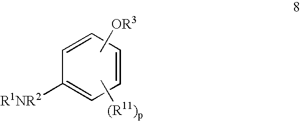
wherein R¹, R², R³, R¹¹ and p are as defined for formula 1.

3. The method according to claims 1 or 2, wherein R³ is —(CR¹R²)ₜ(4 to 10 membered heterocyclic), wherein t is an integer from 0 to 5, and the foregoing R³ groups are optionally substituted by 1 to 3 R¹⁰ groups.

4. The method according to claim 3, wherein said heterocyclic group is optionally fused to a benzene ring or a C₅-C₈ cycloalkyl group, and the foregoing R³ groups, including any optional fused rings, are optionally substituted by 1 to 3 R¹⁰ groups.

5. The method according to claims 1 or 2, wherein R³ is selected from 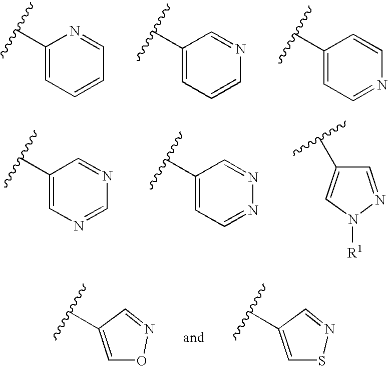
wherein the foregoing R³ groups are optionally substituted by 1 to 3 R¹⁰ groups.

6. The method according to claims 1 or 2, wherein R³ is pyridin-3-yl optionally substituted by 1 to 3 R¹⁰ groups.

7. The method according to claims 1 or 2, wherein R⁴ and R⁵ are both hydrogen.

8. The method according to claims 1 or 2, wherein R¹³ and R¹⁴ are both hydrogen.

9. The method according to claims 1 or 2, wherein R¹⁵ and R¹⁶ are both hydrogen.

10. The method according to claims 1 or 2, wherein k is 1.

11. The method according to claims 1 or 2, wherein l is 1.

12. The method according to claims 1 or 2, wherein R¹⁷ is a t-butyl group.

13. The method according to claims 1 or 2, wherein R¹⁹ and R²⁰ are both OR¹⁸ wherein each R¹⁸ independently is C₁-C₆ alkyl.

14. The method according to claims 1 or 2, wherein R¹⁸ is a t-butyl group.

15. The method according to claim 1, wherein R¹⁹ is —(CR¹⁵R¹⁶)ₗOR¹⁷ and R²⁰ is OR¹⁸.

16. The method according to claim 1, wherein X is a halide selected from the group consisting of chloride, bromide and iodide.

17. The method according to claim 1, wherein the catalyst is palladium or nickel catalyst selected from the group consisting of palladium on carbon (Pd/C), Pd(OAc)₂, Pd₂(dba)₃, PdCl₂, Pd(MeCN)₂Cl₂, Pd(PhCN)₂Cl₂, PdCl₂(PPh₃)₂, Pd(PPh₃)₄, BnPdCl(PPh₃)₂, Pd(Otfa)₂, Pd(PPh₃)₂(Otfa)₂, PdCl₂(dppf), Pd(acac)₂, Pd₂(dba)₃-CHCl₃, Ni(PPh₃)₄, Pd(dppb), trans-di(μ-acetato)-bis[o-(di-o-tolylphosphino)benzyl]dipalladium(II), bis(1,3-dihydro-1,3-dimethyl-2H-imidazol-2-ylidene)diiodo-palladium and diiodo[methylenebis[3-(2-methyl)-1H-imidazol-1-yl-2(3H)-ylidene]]-palladium.

18. The method according to claim 1, wherein said ligand is selected from the group consisting of a polymer bound phosphine, BINAP, dppf, 2-methyl-2′(dicyclohexylphosphino)biphenyl, 2-dimethylamino-2′-(dicyclohexylphosphino)biphenyl, and P(R²²)₃, wherein each R²² is independently selected from the group consisting of 2-methyl-2′-(dicyclohexylphosphino)biphenyl, 2-dimethylamino-2′-(dicyclohexylphosphino)biphenyl, phenyl, o-toluyl, OMe, and furyl.

19. The method according to claim 1, wherein the base is wherein said base is selected from the group consisting of (R)₃N, (R)₂NH, RNH₂, QX, Q₂CO₃, Q₃PO₄, QO₂CR, wherein Q is selected from the group consisting of (R)₄N, Na, K, Cs, Cu, Cd, and Ca, and wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are independently selected from H and C₁-C₆ alkyl.

20. The method according to claim 1, wherein said reaction is carried out in a solvent selected from the group consisting of toluene, benzene, xylene, dimethylformamide, dimethylacetamide, dioxane, tetrahydrofuran, acetonitrile, N-methylpyrrolidinone, dimethylsulfoxide, dimethoxyethane, CH₂Cl₂, CHCl₃, ClCH₂CH₂Cl, N(C₁-C₆ alkyl)₃, N(benzyl)₃, HO(C₁-C₆ alkyl), acetone, methylethylketone, methylbutylketone, and mixtures thereof.

21. The method according to claim 20, wherein said HO(C₁-C₆ alkyl) is 2-propanol, 2-butanol, or a mixture thereof.

22. The method according to claim 1, wherein said reaction is carried out at a temperature ranging from about 25° C. to about 175° C.

23. The method according to claim 1, wherein the compound of formula 2 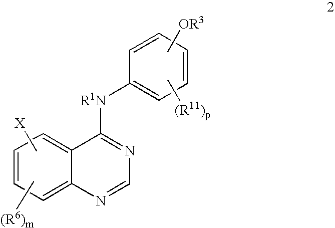
is prepared by reacting a compound of formula 2A 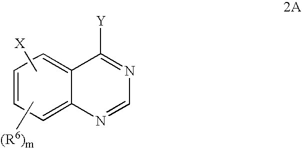
wherein Y is a halide and X, R⁶ and m are as defined for formula 1, with a compound of formula E 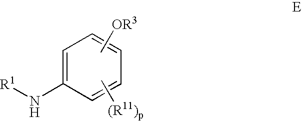
wherein R¹, R³, R¹¹, and p are as defined for formula 1.

24. The method according to claim 1, further comprising converting the compound of formula 1 in one or more steps to produce a compound of formula 5 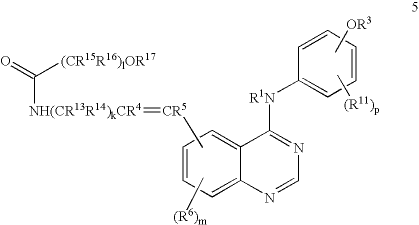
wherein R¹, R³, R⁴, R⁵, R⁶, R¹¹, R¹³, R¹⁴, R¹⁵, R¹⁶,R¹⁷, k, l, m, and p are defined for formula 1 in claim 1.

25. The method according to claim 24, wherein the compound of formula 5 is selected from the group consisting of:
E-2-Methoxy-N-(3-{4-[3-methyl-4-(6-methyl-pyridin-3-yloxy)-phenylamino]-quinazolin-6-yl}-allyl)-acetamide;
E-N-(3-{4-[3-Chloro-4-(6-methyl-pyridin-3-yloxy)-phenylamino]-quinazolin-6-yl}-allyl)-2-methoxy-acetamide;
E-N-(3-{4-[3-Chloro-4-(6-methyl-pyridin-3-yloxy)-phenylamino]-quinazolin-6-yl}-allyl)-acetamide;
E-2-Ethoxy-N-(3-{4-[3-methyl-4-(6-methyl-pyridin-3-yloxy)-phenylamino]-quinazolin-6-yl}-allyl)-acetamide;
E-N-(3-{4-[3-Methyl-4-(6-methyl-pyridin-3-yloxy)-phenylamino]-quinazolin-6-yl}-allyl)-methanesulfonamide;
and the pharmaceutically acceptable salts, prodrugs and solvates of the foregoing compounds.

26. The method of claim 24 wherein converting the compound of formula 1 to the compound of formula 5 comprises the steps of:
(a) reacting the compound of formula 1 with an acid to form a compound of formula 4 or a salt thereof 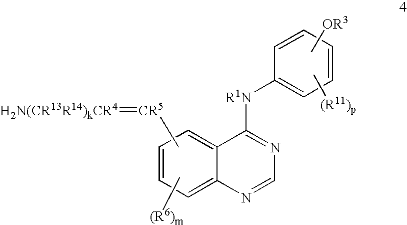
and (b) reacting the compound of formula 4 or its salt with ClC(O)(CR¹⁵R¹⁶)ₗOR¹⁷ or a reactive equivalent thereof in the presence of a base to form the compound of formula 5.

27. The method according to claim 26, wherein in step (b), the reactive equivalent of ClC(O)(CR¹⁵R¹⁶)ₗOR¹⁷ is an acid imidazole represented by the formula 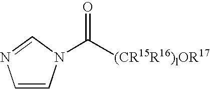
or an acid anhydride represented by the formula [R¹⁷O(CR¹⁵R¹⁶)ₗC(O)]₂O.

28. The method according to claim 26, wherein in step (b), the base is at least one compound selected from the group consisting of an aqueous hydroxide of an alkali or alkaline earth metal, a carbonate, phosphate or hydrogen phosphate of an alkaline earth metal, an tertiary amine and DABCO.

29. The method according to claim 26, wherein step (b) comprises reacting the compound of formula 1 with an acid in one step to produce the compound of formula 5.

30. A method for preparing a compound represented by the formula 3a 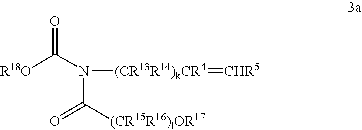
wherein R⁴ and R⁵ are independently selected from hydrogen and C₁-C₆ alkyl; each R¹³, R¹⁴, R¹⁵ and R¹⁶ is independently selected from hydrogen, C₁-C₆ alkyl and CH₂OH; and R¹⁷ and R¹⁸ are C₁-C₆ alkyl; and k and l are independently 1 to 3, comprising the steps of:
(a) reacting an amine represented by the formula H₂N—(CR¹³R¹⁴)ₖCR⁴═CHR⁵ wherein R¹³, R¹⁴ and k are as defined for formula 3a, with a compound represented by the formula R¹⁷O(R¹⁶R¹⁵C)ₗC(O)X where X is a halide, or a reactive equivalent thereof to form a compound represented by the formula 6 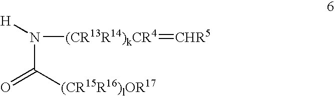
and (b) reacting the compound represented by the formula 6 with a compound of formula (R¹⁸OC(O))₂O or a reactive equivalent thereof optionally in the presence of a basic catalyst to form the compound represented by the formula 3a.

31. The method of claim 30, wherein the basic catalyst is dimethylaminopyridine (DMAP).

32. The method of claim 30, wherein R⁴ and R⁵ are both hydrogen.

33. The method of claim 30, wherein R¹³, R¹⁴, R¹⁵ and R¹⁶ are all hydrogen.

34. The method of claim 30, wherein k and l are both 1.

35. The method of claim 30, wherein R¹⁷ is methyl and R¹⁸ is t-butyl.

36. A compound represented by the formula 3a 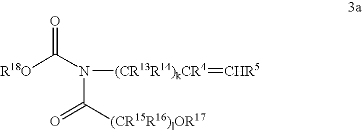
wherein R⁴ and R⁵ are independently selected from hydrogen and C₁-C₆ alkyl; each R¹³, R¹⁴, R¹⁵ and R¹⁶ is independently selected from hydrogen, C₁-C₆ alkyl and CH₂OH; and R¹⁷ and R¹⁸ are independently C₁-C₆ alkyl; and k and l are independently 1 to 3.

37. The compound according to claim 36, wherein R⁴ and R⁵ are both hydrogen.

38. The compound according to claim 36, wherein R¹³, R¹⁴, R¹⁵ and R¹⁶ are all hydrogen.

39. The compound according to claim 36, wherein k and l are both 1.

40. The compound according to claim 36, wherein R¹⁷ is methyl and R¹⁸ is t-butyl.
